(19) United States
(12) Patent Application Publication
(10) Pub. No.: US 2023/0091973 A1
(43) Pub. Date: Mar. 23, 2023

(54) Medzone.

(71) Applicant: Amar Chowdry, Teaneck, NJ (US)

(72) Inventor: Amar Chowdry, Teaneck, NJ (US)

(21) Appl. No.: 17/197,011

(22) Filed: Mar. 9, 2021

Related U.S. Application Data
(60) Provisional application No. 62/987,155, filed on Mar. 9, 2020.

(51) Int. Cl. G06Q 30/0601; G16H 40/20; G16H 20/10

(52) U.S. Cl. CPC G06Q 30/0639; G16H 40/20; G06Q 30/0641; G16H 20/10

(57) ABSTRACT

According to an aspect of present disclosure, a method performed on a computer system comprises receiving a geographical location, determining a geographical boundary in relation to the geographical location, listing a set of medical service providers within the boundary from a plurality of medical service providers, displaying the set of medical service providers for selection, selecting a first medical service provider in the set of medical service providers and connecting to the first medical service provides, wherein said connecting comprising one establishing communication, placing order, making an appointment, booking a facility.

### FIELD OF INVENTION

The present invention relates generally to the field of Information Technology (IT) and more particularly to a means for a consumer to connect with a healthcare provider of necessity to them such as a pharmacy, urgent care and hospital ER (Emergency Room).

### RELATED ART

The prevailing model for e-commerce is that the seller/vendor or service provider is offering items or service for sale without any direct interaction with the buyer.

### SUMMARY

According to an aspect of present disclosure, method performed on a computer system comprises receiving a geographical location, determining a geographical boundary in relation to the geographical location, listing a set of medical service providers within the boundary from a plurality of medical service providers, displaying the set of medical service providers for selection, selecting a first medical service provider in the set of medical service provider and connecting to the first medical service provider, wherein said connecting comprising one of a establishing communication, placing order, making an appointment, booking a facility.

According another aspect, the computer system comprising a mobile device, a computer, a database, first server, a GIS server and an AI server, wherein the database comprising a plurality of data sets corresponding to the plurality of medical service providers, in that, each data set comprising at least one of a location information, services offered, stock of medicine, facilities, inventory, product information, and specialization. The plurality of medical service providers are grouped under plurality of categories comprising at least one of Pharmacy, doctors, hospital, laboratory, urgent care, harm reduction.

According to another aspect the method further comprises providing a user interface for searching a medicine, presenting a list of preferred medicine, a text entry filled to enter the medicine name, selecting a first medicine from at least one of the list of preferred medicine and result of a search and listing a set of pharmacies having the first medicine in stock and located within the boundary, selecting a first pharmacy from the set of pharmacies, placing an order for the first medicine with the first pharmacy, and managing the order, said managing comprising at least one of a sending an order details to the first pharmacy, facilitating delivery of medicine.

According to yet another aspect the method comprises providing a user interface for searching a first medical service provider in one of said category, providing a list of preferred service providers, presenting a list of category of medical service provider indicating a number of medical service providers within the geographical boundary, selecting a first medical service provider category from the list of category, presenting the first list of medical service providers of first category that are within the geographical boundary, selecting a first medical service provider at least from the first list or from the list of preferred medical service providers, placing a service request with first medical service provider, wherein said placing includes the selection of date, service type and location of service and booking the service request with the first medical service provider.

According to yet another aspect the method comprises providing a user interface for searching a doctor, providing a list of preferred doctor, presenting a list of doctors within the geographical boundary, presenting the list of doctors on a map, selecting a first doctor in the list of doctors, presenting the details of the first doctor, placing an appointment request with the first doctor and recording a successful completion of the appointment.

According to yet another aspect a networked computer systems operative to provide medical services to a user comprises at least one mobile device configured to operate as user device, a plurality of computers configured to operate as corresponding plurality of service providers, a medical service facilitation server to facilitate medical services between the plurality of computers and the mobile device, an Artificial intelligence server configured to perform tasks by applying logical reasoning that is similar to human logical reasoning, a Machine learning engine configured to modify rules and conditions applied by the medical service facilitation server and Artificial intelligence server, a GIS server operative to determine the location of the mobile device and a database configured to store datasets comprising at list one of products, services, facilities, availability, price, inventory, address, location, access information and any other information relevant for the services offered by the respective service providers and a map server providing a first map in relation to the location of the mobile device wherein, the medical service facilitation server presents a list of service providers on the mobile device based on the location of the mobile device.

According to another aspect, the networked computer systems comprises a location based health care/service provider module 410 operative to provide location based service provider access, a medicine search module 420 operative to search medicine and its availability with the service providers, a rating module 430 operative to receive reviews and provide ratings of the service provider, a search doctor module 440 operative to search doctors by specialization and location, a suggestion module 450 operative to generate suggestion and present the suggestion along with other services and a appointment booking module 460 operative to book appointments and facilities and monitor the same.

### BRIEF DESCRIPTION OF DRAWINGS

FIG. 1 is a block diagram illustrating an environment in which various aspects of the present invention may be seen

FIG. 2 is a block diagram illustrating an example embodiment of Geo-location based medical and health service access system deployed in the environment.

FIG. 3 illustrates plurality of data sets received and stored in the database by the services providers.

FIG. 4 illustrates set of example functional elements/modules of the Medzone.

FIG. 5 is a flowchart illustrating the manner in which the module may be implemented in an embodiment.

FIG. 6 is a flowchart illustrating an example deployment of block in one embodiment.

FIG. 7 is example software (configuration) implementing the modules and elements.

FIG. 8A illustrates an example user interface for receiving user location.

FIG. 8B illustrates the example user interface for displaying plurality of categories of service providers on the device.

FIG. 8C illustrates alternative user interface for displaying plurality of category of service providers on the device.

FIG. 9A and 9C illustrate the manner in which details of the service providers are presented on mobile device.

FIG. 10A and 10B illustrate an example user interface on the device for searching medicine.

FIG. 11A-11D illustrate the user interface on the device enabling the search of doctors.

FIG. 12A and 12B illustrate the user interface for booking appointment and the confirmation respectively.

FIG. 13 and 14 illustrate the review and review input user interface respectively.

### DETAILED DESCRIPTION OF THE PREFERRED EXAMPLES

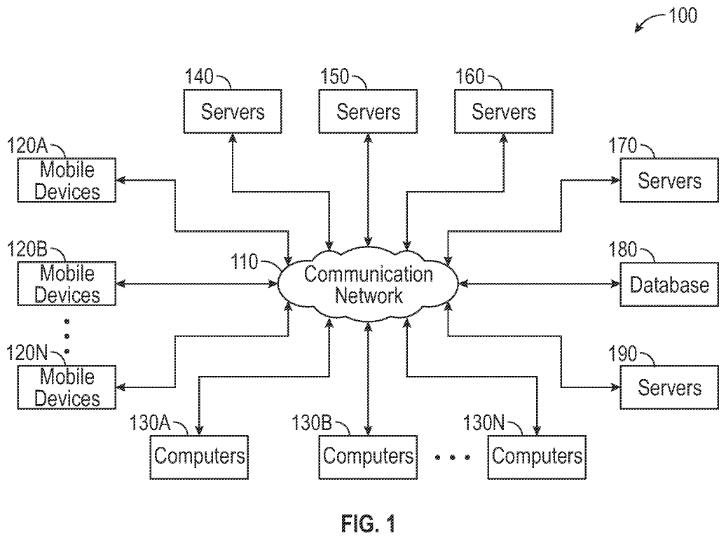

FIG. 1 is a block diagram illustrating an environment in which various aspects of the present invention may be seen. The environment 100 is shown comprising communication network 110, mobile devices 120A-N, computers 130A-N, Artificial intelligence (AI) server 140, Machine learning (ML) server 150, global information system (GIS) 160, Map server 170, database 180, and medical zone server (Medzone) 190. Each element is described in further details below.

The communication network 110 communicatively connects the elements (120-195) of the environment 100. The communication network 110 comprises plurality of communication channels for exchanging information and data by and between one or more elements (120-190). The channels may comprise a short distance channel, long distance channel, secured channel, unsecured channel, shared channel, private channel, dedicated channel, encrypted channel and combinations thereof, for example. The communication network 110 may deploy the channels for communication over a wired link, wireless link, satellite link and combinations thereof. The communication network 110 may comprise networked communication means employing several standard protocols for multi-devices connectivity, simultaneous connectivity, coordinated and cooperated connectivity so that group of devices may participate in sharing and exchanging data among the known group. The networked communication means may comprise internet, GSM, Ethernet, Bluetooth, LAN, WAN, (www) wordwideweb, ZigBee, WI-FI, 5G networks, 4G networks, 3G networks, 2G networks, WiMAX, CDMA, GSM and such. Each term therein taking the respective meaning as is weal known an the relevant art. The networked communication means may comprise one or more communication channels.

The mobile devices 120A-N perform several operations to provide desired functionality. The mobile devices 120A-N may comprise operational elements: a digital processor for executing digital instruction, program, software application; a memory for storing data and digital instructions; input device for receiving data, commands and inputs from a user; display device for displaying text data, graphical data, images, video data; audio devices for receiving and providing audio information; modems (Modulator and demodulators) for establishing communication with communication channel 110; GPO device to determine the location of the mobile device; inertial navigation sensors (INS) to determine position, angle acceleration etc., for navigation and recording movements of the mobile device; peripheral interfaces for connectivity to external devices such as printer, scanner, power charger; and a battery for powering the mobile device. The mobile devices (Generally referred to as 120) employ one or more operational elements to provide the desired functionality. The mobile device 120 may be deployed with an operating system such as IoS and Android to enable deployment of functionality by way of mobile application (referred to as Mobile App). The mobile app (set of digital instruction) when executed by the Processor, is configured to interact with the operational elements in a specific manner to provide unique functionality and utility.

The computers 130A-N are computing devices comparably with more computing powers than the mobile devices. The Computers 130A-N may operate as terminal devices, devices, client device, internet browsing device, distributed computing device, etc., in a one or more configurations of master-slave, server-client, networked servers, terminal device etc. The computers 130A-N may be deployed with an operating system and an internet browser to connect and access, one or more servers, the services, applications, databases connected on the communication network 110. Further, computers 10A-N may also be deployed with a client application, slave application, stand alone applications that access the services, applications, databases connected on the communication network 110. The computer 130A-N may comprise the operational elements similar to the ones disclosed in respect of mobile devices 120A-N.

The Artificial intelligence server 140 performs tasks applying logical reasoning that is similar to human logical reasoning (referred to as artificial intelligence (AI)) based on the current and past data. The artificial intelligence server 140 is connected to the communication network 110. The artificial intelligence server may access data from the other elements 120-190 of the environment 100 for performing the Al tasks. The results and tasks are provided to one or more elements of the environment 100, In other words, the other elements 120-190 may access the service of the artificial intelligence server 140 to determine the environment behaviour, user preferences, possible next action, forecasting, market behaviour, user behaviour, etc. The artificial intelligence server 140 may be configured with the number of application interfaces protocols (referred to as APIs) that are made available to the other elements 120-190 for accessing the services and the server 140. In certain embodiment, the artificial intelligence server 140 in may be deployed distributively on multiple devices including the mobile devices 120 and computers 130 so that the AI tasks are partially executed on the mobile device and computer device. In certain embodiment, the artificial intelligence server 140 may be a constellation of set of servers operating under several technologies that allows machines to sense, comprehend, act and learn in order to extend human capabilities. The plurality of technologies may comprise, image processing', natural language processing, voice processing, virtualisation, process automation, data analytics, predictive analytics, image recognition, video recognition, object detection, face detection, statistical modelling , block chain capabilities for example. In certain embodiment , the artificial intelligence server 140 may be deployed to operate in conjunction with the Machine learning engine 150. The artificial intelligence server I 40 may comprise a plurality of computer machines together operative as a server (together executing instructions that perform Al task).

The Machine learning engine 150 generates new rule, modifies existing rules and conditions based on the current and history data. The elements of the environment 100 may adopt one or more rules and conditions (also referred to as algorithms) that are dynamically modified by the machine learning engine 150. In certain embodiments, the artificial intelligence server 140 may dynamically adopt the rules and conditions as modified by the machine learning engine 150 in performing AI tasks. In certain embodiment, the machine learning engine may be configured to identify relevant data and prepare the data for analysis select and adopt one or more algorithms (such as XG roost, Logistic regression and Random forest) to perform data analysis build analytic models, train and test data sets and models run the models to determine the score. In that the models representing the rules and conditions. In one embodiment the artificial intelligence server 140 and the machine learning engine 150 may operate together to generate several data feeds to other elements 120-190 in general and to the mob le devices 120 and computers 130 in particular. The machine learning engine 150 may comprise a plurality of computer machines deployed over multiple locations connected together through communication network 110. In certain embodiment, the machine learning engine may be deployed distributively over the mobile device 120 and computer 130.

The global information system (GIS) 160 provides spatial data and other data units that are associated spatially to geographical locations. In certain embodiment, the GIS 160 may be operative to perform operations of capturing, storing, analysing, manipulating, and displaying data units that are associated to spatial locations. In certain embodiment, the GIS 160 may retrieve/receive maps from the map server 170 and associate the data units to one or more one or more maps. Elements 120-190 may access the GIS 160 retrieve spatial information, results of demographic analysis, data associated with locations etc.

The Map server 170 provides the geographical map with one or more layers of information/data superimposed on the maps. In certain embodiment, the one or more layer of information may comprise terrain, roads, structures, landmarks, contours, boundaries, area, city, natural resources, shops, business houses, service centers, business establishments, houses, residential layouts, boundaries of country, state, district, locality, etc, for example. In certain embodiment, The Map server 170 may update maintain the records of the maps provided to the elements 120-190 and update the maps in the devices that are connected to the communication network 110. The GIS 160 and map server 170 may receive, time to time, additional geographical information comprising conditions such as traffic, goods/material availability at specific location, user location, user activity at a specific location etc., from the mobile device 120, computers 130, the AI server 140, ML machine 150 and medical zone server (Medzone) 190.

The database 180 stores the data in the environment. In that, the database 180 may comprise plurality of databases that are specifically associated with the elements mobile device 120, computers 130, the AI server 140, the ML engine 150, the GIS 160, the map server 170 and Medzone 190. Further, the database 180 may also comprise shared data bases (also referred to as data storage) that are commonly accessed by one or more elements of the environment 100. Further, the database 180 also comprises data stored in The mobile devices 120, computers 130, AI server 140, ML engine 150, GIS 160, map server 170 and Medzone 190. In certain embodiment, The database 190 is plurality of data storages and data warehouses with the access control distributed globally over multiple locations and interconnected together through communication network 110.

The medical zone server (Medzone) 190 provides a geographical location (geo-location) based medical and health care service access and management on the environment 100. In that, the elements 120-190 are configured and operative to enable a user of the device 120 and 130 to access, avail, offer, render, and make available one or more medical/health care services. The manner in which the environment 100 may be configured to provide a geo-location based health and medical service access is described in further detail below.

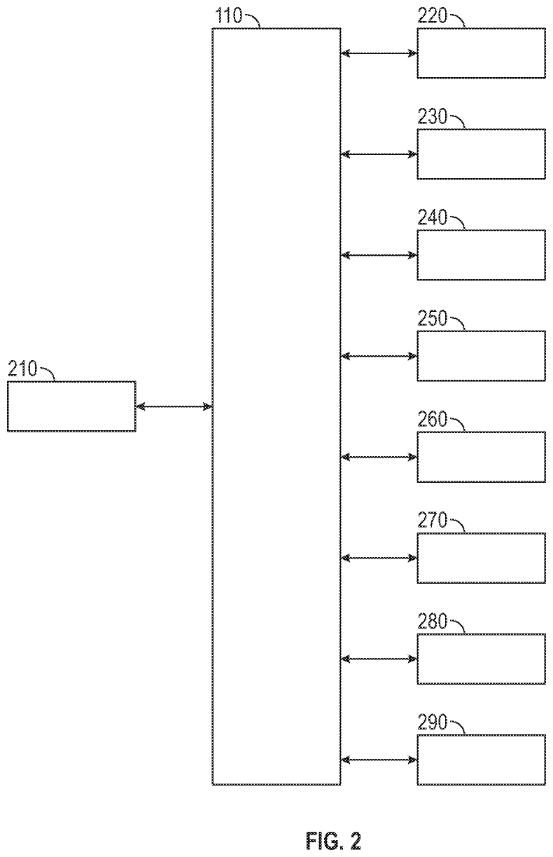

FIG. 2 is a block diagram illustrating an example embodiment of Geo-location based medical and health service access system 200 deployed in the environment 100. The block diagram is shown comprising patient 210, pharmacy 220, hospital 230, nursing home 240, doctor 250, nurse 260, clinic 270, special health care services 280, and health program centers 290.

In that, the pharmacy 220, hospital 230, nursing home 240, doctor 250, nurse 260, clinic 270, special health care services 280, and health program centers 290 representing the examples of types of devices operative by (and as) the health care service providers. The patient 210 representing an example device operative by (or as) a health care service user (in general referred to as user). User 210 interacts with the service providers 220-290 over the communication channel 110.

Each health care service provider device (220-290) is configured to receive the request for medical products/services at least from the patient unit 210. Further, the devices (220-290) are configured to create and maintain dataset comprising at least one of products, services, facilities, availability, price, inventory, address, location, access information and any other information relevant for the services offered by the respective service provider devices (220-290).

PA For example, but not limited to the pharmacy 220 may create and maintain a pharmacy data comprising list of products, availability, price, inventory, address, location, access information. Besides address, location, access information, the hospital 230 may create and maintain a hospital facilities details comprising list of beds, specialty, operation theatres, Emergency facility, doctors, nurses, ambulance services, availability, price, inventory. The doctor 250 may create and maintain a doctor details comprising but not limited to, specialization, profile, availability schedule, and price. Similarly, other service provider devices may be configured to create and maintain the datasets specific to the respective services. In certain embodiment, the service provider devices 220-290 are deployed on a computer (130)/mobile device (120) as an application. In that, respective devices are configured to upload and maintain the respective service provider data sets in the database 180.

The patient (unit_210 is configured to enable a patient (also referred to as customer/user) to access the health care (medical) services and health care products from the providers (220-290). In certain embodiment, the patient unit 210 is deployed on a mobile device (120) as an application. In that, the patient unit 210 determines the location of the mobile device to present a list of health care service providers within a geographical boundary for accessing. The patient unit 210 is further configured to display the list of service providers within the geographical boundary. The patient unit 210 is further configured to display the location of the service providers on a Geographical map. In that, the patient unit 210 may display position of the patient unit 210 in relation to the service provider locations either graphically or by text. In certain embodiments, the patient unit 210 may be configured to display medical products such as medicine, drugs and other medical accessories derived from the data sets of the services providers such as pharmacy 220. In certain embodiment, the patient 210 and service provider device 220-290 interact between each other in order to ensure the requested services are rendered to the patient.

In one embodiment, the Medzone 190 manages and facilitates the operations of the patient 210 and service providers 220-290. In that, the patient 210 and service providers 220-290 may connect with the Medzone 190 to send/receive data, service request, status, locations, maps, etc. The Medzone 190 may receive the service provider data and the patient data and store in the database 180. The Medzone 190 may connect with elements 140-170 to avail the respective services to facilitate the communication between the patient 210 and the service providers 220-290.

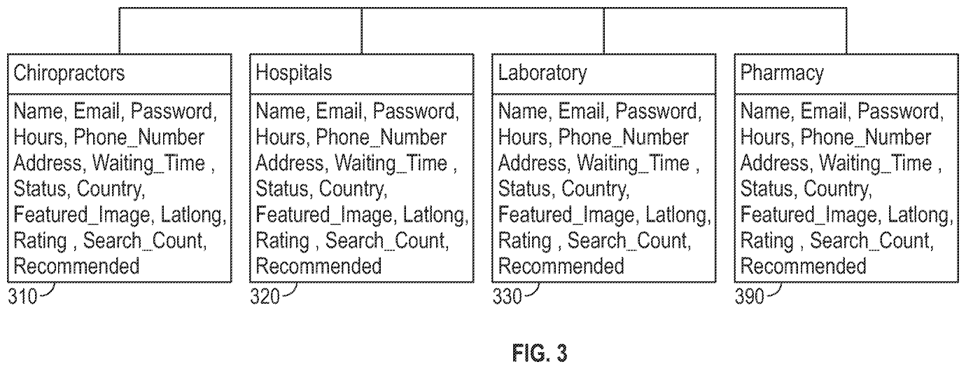

FIG. 3 illustrates plurality of data sets 300 received and stored in the database by the services providers 220-290. The plurality of data sets 300 is shown comprising data sets 310-390. Each data set may comprise plurality of interconnected/independent data tables. The data set 310 represents the data of the chiropractors/doctors uploaded and maintained on the data base 180. The data set 320 represents the data of the hospitals uploaded and maintained on the data base 180. The data set 330 represents the data of the laboratory service provider uploaded and maintained on the data base 180. The data set 390 represents the data of the pharmacy.

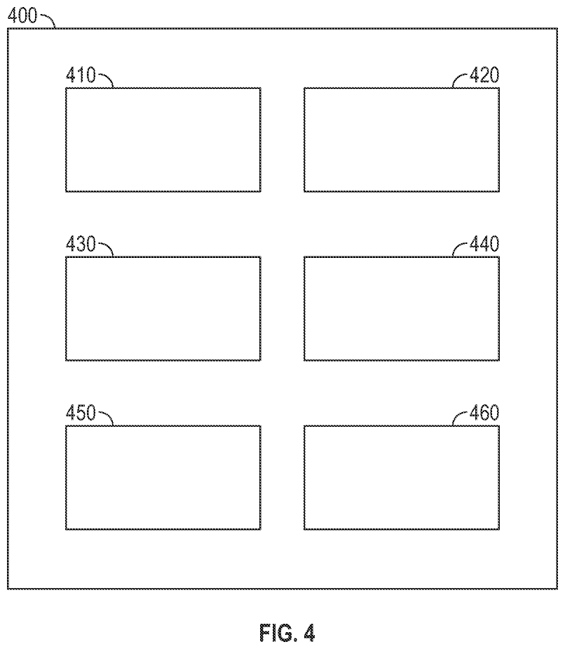

In one embodiment, the Medzone 190 may comprise plurality of functional elements/modules. FIG. 4 illustrates set of example functional elements/modules of the Medzone 190. As shown there, the Medzone 400 comprises: the location based health care/service provider module 410 that is operative to provide-location based service provider access; the medicine search module 420 operative to search medicine and its availability with the service providers; the rating module 430 operative to receive reviews and provide ratings of the service provider; the search doctor module 440 operative to search doctors by specialization and location; the suggestion module 450 operative to generate suggestion and present the suggestion along with other services; and the appointment booking module 460 operative to book appointments and facilities and monitor the same.

In certain embodiments, the modules 410-460 are deployed as software application/programs executable on the mobile devices 120, computers 130, Medzone 190. In certain other embodiment, the software application and programs may be distributively executed partially on mobile device and partially on other elements 120-190 of the environment 100. The manner in which example modules are operative is further described below.

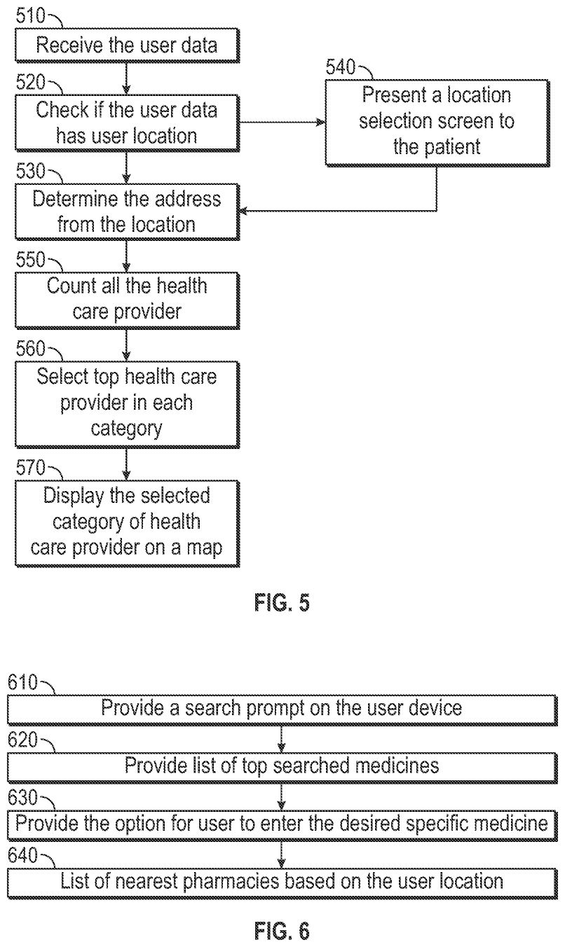

FIG. 5 is a flowchart illustrating the manner in which the module 410 may be implemented in an embodiment. In the block 510, Medzone 190 may receive the user data. The user operating the mobile device or computer that is configured as patient 210 may receive user data through keyboards and other input devices. In an embodiment, device may be configured to prompt user to input the user data using input device. In yet another embodiment, the device may automatically determine its location through GPS and INS sensors and provide the location information in the form of latitude and longitude. In yet another embodiment, the user device 210 may coordinate with the GIS and Map servers and provide the location information and address to the Medzone 190. In yet another embodiment, the user device may register the user with the Medzone and may be operative in a collaborated manner such that user data may be received through registration, login credentials etc.

In the block 520, Medzone 190 may check if the user data has user location. If the user location is present in the user data, the control passes to block 530, else to block 540. For example, in the event user device is disabled to determine its location, the user/device is not registered with Medzone 190 and the user input does not contain the address/location information.

the block 530, the Medzone 190 may determine the address from the location. For example, when the user data provided in the block 510 comprises the location information, where the location information is in the form of latitude and longitude, the medzone may determine the address corresponding to the received location by interacting with the GIS 160 and map services 170.

In the block 540, Medzone may display a location selection screen to the patient 210 enabling the patient to select a location. In one embodiment, the medzone may embed/incorporate the services map server 170 and GIS 160 into the user device 210/120. A map with ability to select a location is presented on the user device 120/130. The device is enabled either through input device and display for example such as touch screen display for user to touch the location on the displayed map for making a selection. The selection so made is provided to Medzone 190 as the user location.

In one embodiment, the data received in the block 510 and 540 may additionally comprise medical service type/category. For example, the user may, in addition to location and address information, specify the type of medical services such as pharmacy, doctor, clinic, Hospital ER room, etc. In an yet another alternative embodiment, the artificial intelligence 140 and ML engine 150 may determine the medical service category most likely desired by the user based on the user history data, time of request, location request etc.

In the block 550, Medzone 190 may count all the health care providers of a specified category within a geographical boundary. The Medzone may determine the geographical boundary based on the location and/or address information received in the block 510 and 540. As an example, the Medzone may select the boundary as an area within 2 miles of radius from the received location. In an alternative embodiment, the Medzone may determine the boundary as area that is within 30 Mins drive from the location. On selection of the boundary or area covering the boundary, the Medzone determines the list of health service providers in the selected area. The list or health care service providers are determined from the data sets uploaded by each service provides 220-290. Further, the Medzone may select the service provider of a particular category that is specifically requested or determined as relevant to the user.

In the block 560, Medzone 190 may select top health care provider in each category. In that, the medzone may fetch the review and rating data of each service provider and determine the top service provider as the ones having ranks above a threshold (for example having rank above 4 stars). Alternatively, the Medzone may arrange the health care service providers in the descending order of ranks and select top two service providers.

In block 570, Medzone displays the selected category of health care providers on a map. In block 580, the Medzone provides details and reviews of top health care provider. For example, the Medzone may display both map and the list of service providers side by side. Alternatively, Medzone may display the service providers on the map and show list of the service providers in response to user prompt. Further, Medzone may be configured to show the top service provider with reviews and details to draw more attention towards the top service providers.

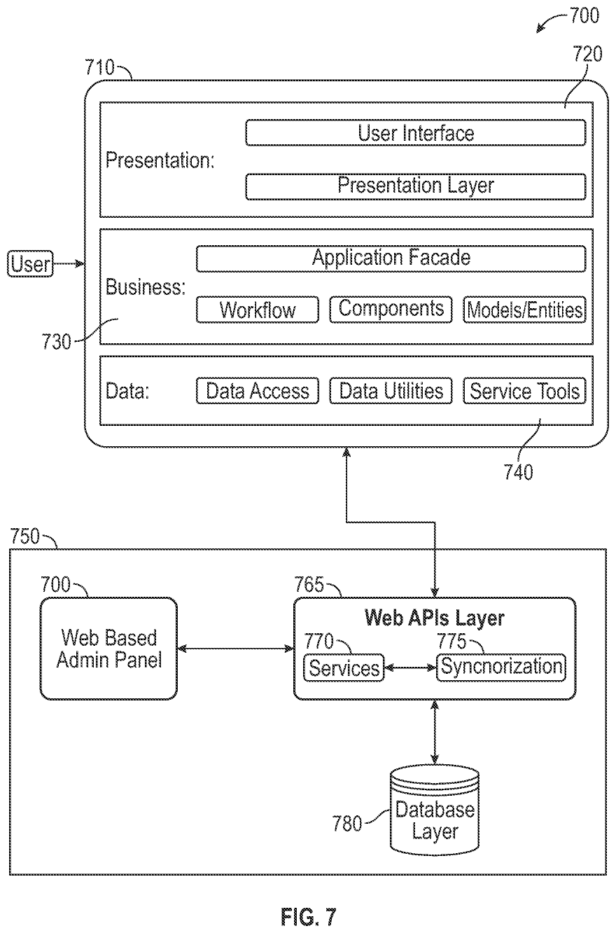

The manner in which the module 420 may be implemented is further described. FIG. 6 is a flowchart illustrating an example deployment of block 420 in one embodiment. In that, in the block 610, Medzone 190 provides a search prompt on the user device 120/210. The search prompt may be provided as a selectable button on the display screen for user to select/touch the button by means of input devices. Medzone may monitor the user activity action to determine the user selection. In one embodiment, the search prompt may be associated to search medicine. On determining user(s) desire to search, in block 620, Medzone provides list of top searched medicines. In block 630, Medzone provides the option for user to enter the desired specific medicine. In one embodiment, the list of top searched medicines and the option for user to enter the specific medicine are provided side by side. In one embodiment the Medzone may determine the top searched medicine based on the recommendations received from the Al server 140 and ML engines 150. In that, the Medzone may maintain search history of all users to determine the top searched medicine. Alternatively, Medzone may receive recommendation from the Al and ML engines to present the most searched medicine. Wherein the AI server may determine the most searched (desired) medicine based on the time, location, user history, current medical scenario in the locality/country etc. Device 210 may enable user to select a medicine from the top searched list of medicine or from the result of the search. The selection of the medicine is received as desired medicine.

In block 640, Medzone 190 provides list of pharmacies that are nearest to the user location. The list of pharmacy may be arranged on the display based on the ratings, distance, geographical boundary etc. In one embodiment, the Medzone may determine the availability of the desired medicine in quantity, status of the pharmacy at the time of request as to closed or open, facility offered by the pharmacy as to availability of delivery service (that are made available as part of the data set of the respective pharmacy).

Continuing with FIG. 4, the rating module 430 may be configured to provide a user interface to receive a review/rating when user avail services of the service provider 220-290. For example, on successful completion: of placing a request for a medicine, consultation of a doctor, on booking a hospital ER room, the rating module may display on the user device a review collection fields for user to enter the data. The reviews and ratings so collected may be stored in the database for computation of the overall rating of the service provider and also may be provided to the AI server 140 and ML engine 150 for analysis. Further, the rating module 430 may be configured to display overall rating on the user device whenever a service provider details are presented/displayed on the user device.

The search doctor module 440 may be implemented similar to the search module 420 described in respect of search medicine described above. Accordingly, the module 440 may facilitate searching doctors of specific specialisation, experience, availability etc. the results may be presented in the similar manner.

The suggestion module 450 is operative to generate suggestion and to present the suggestion along with other services in one embodiment, the suggestion module 450 may determine most desired or best suited services, medicines, doctors, etc through the history data, current data, forecasted data, location, and user details. The most desired/suited services/medicine/doctor etc. are presented on the user device voluntarily, along with any response to the user request, as prompts, notification etc.

The manner in which the appointment booking module 460 is operative to book and monitor appointments, services and facilities may be implemented in an embodiment is further described in one embodiment, the module 460 may facilitate communication between the patient 210 and the selected service provider to book a facility, service and/or consultation. In one embodiment the module 460 may be activated when the user selects a service provider in any one of the modules described above. On selecting a service provider, For example, a doctor, the module 460 presents the details of the doctor, the availability, schedule, price etc. on the user device, for selection. The user may select the time slot through any input device to indicate the choice and submit the booking request. The module 460 is further configured to generate the reference number corresponding to the booking for confirmation and same is communicated on the respective service provider device (220-290). The module 460 may be further configured to send prompts and reminders to the both patient 210 and service provider about the appointment. The module 460 may also be configured to receive confirmation of successful completion of the appointment. Similarly, the module 460 may be further configured to manage and monitor the facility booking such as booking of a hospital ER and to coordinate placing of order for medicine with the selected pharmacy and delivery of the same.

In one embodiment, the elements 210-290 and modules 410-460 are implemented as software executed on the elements 120-190. In an alternative embodiment the modules and elements are implemented on cloud as a software as a service (SAAS), web application, website, client-server application, master-slave application, mobile applications and combinations thereof. One example deployment of the software is described below.

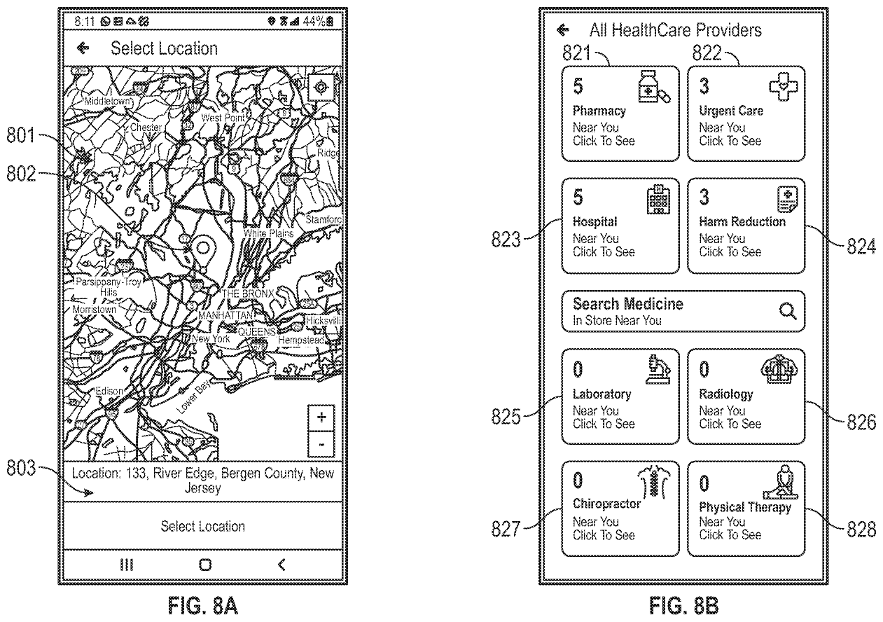

FIG. 7 is example software (configuration) 700 implementing the modules 410-460 and elements 210-290. As shown there, the software 700 comprises mobile application 710, remote infrastructure 750, database 780 and admin panel 790. The mobile application 710 is deployed on the mobile device 120. In that mobile application is operative to provide one or more features described in the sections above in respect of the patient 210. Remote infrastructure 750 is shown comprising web application interface (API) layers 765, services, 770, synchroniser 775. The mobile application 710 interacts with remote infrastructure 750 through web API layers 765. The computers 130 may access the remote infrastructure through services 770. Web API layers 765 and services 770 accesses the data in the database 780 through stored procedures. The admin panel 790 enable control and management of the remote infrastructure. In one embodiment, the mobile application and admin panel may access the remote infrastructure on an encrypted channel. The mobile application is shown comprising: the presentation layer 720 having user interface; the business layer 730 having application facade, workflow, components and model/entities; and the data layer 740 having access data, utility data and service tools. User on the mobile device interacts with the mobile application 710, User on a computer 130 may interact with the services through web browsers. In one embodiment, the features described in respect of FIG. 1 through 6 are deployed in the software configuration 700. Several features operative on the user device 210 are further illustrated below.

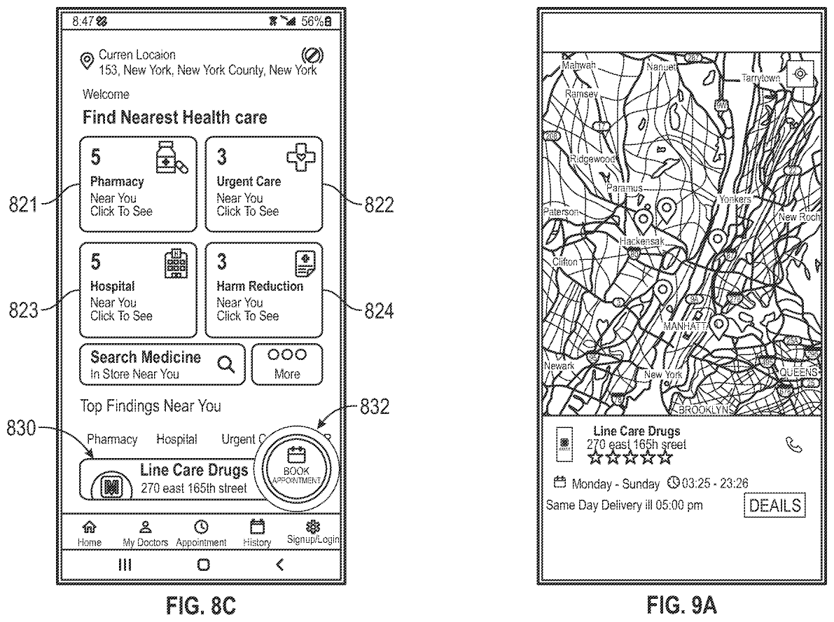

FIG. 8A illustrates an example user interface for receiving user location. As shown there, user device 210 may display a map 801 and a location marker 802. The location marker 802 may be selected and placed on any point on the map to indicate the location input. The address corresponding to the location is displayed as 803.

FIG. 8B illustrates the example user interface for displaying plurality of categories of service providers on the device 210. As shown there, category of service providers 821-828 are displayed with the number of service providers within the boundary. For example, as shown there, number of service provider of the category 801 (pharmacy) within the boundary is 5. Similarly, number of service providers of category 825 (laboratory) is zero.

FIG. 6C illustrates alternative user interface for displaying plurality of category of service providers on the device 210. As shown there, category of service providers 821-824 are displayed along with the recommended service provider 830. Further, a clickable button 432 providing interface to appointment booking module 460 is provided. In the user interface of FIG. 8B and 8C user may select the specific category of service provider to get more details.

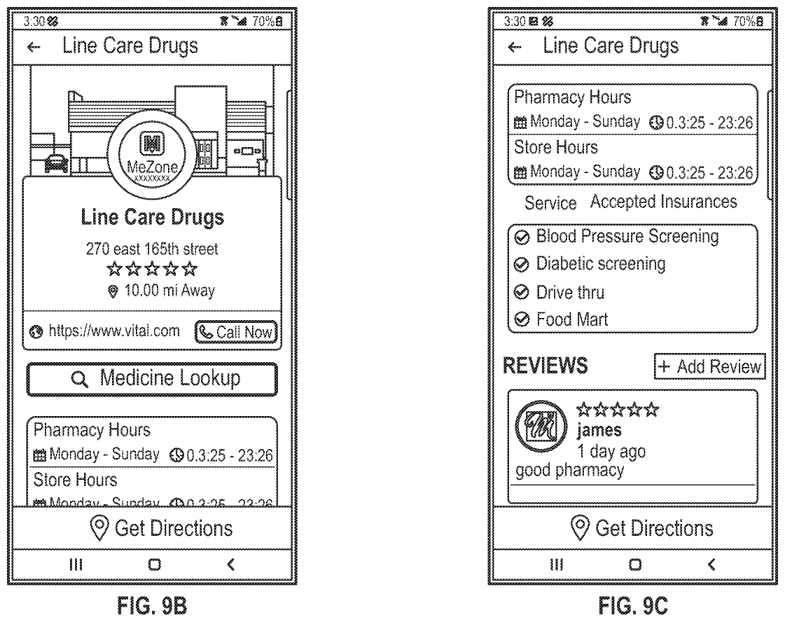

FIG. 9A and 9C illustrate the manner in which details of the service providers are presented on mobile device 210. Once user selects one of the category in the user interface of FIG. 8B and 8C, the list of service providers (five pharmacies) are provided on a map as in FIG. 9A. As shown, the five pharmacies within the geographical boundary are showed on the map and also the details of the five pharmacies are listed below the map that may be scrolled down. User may select any of the five pharmacies for further details FIG. 9B and 9C illustrate example user interface displaying the details of the selected pharmacy.

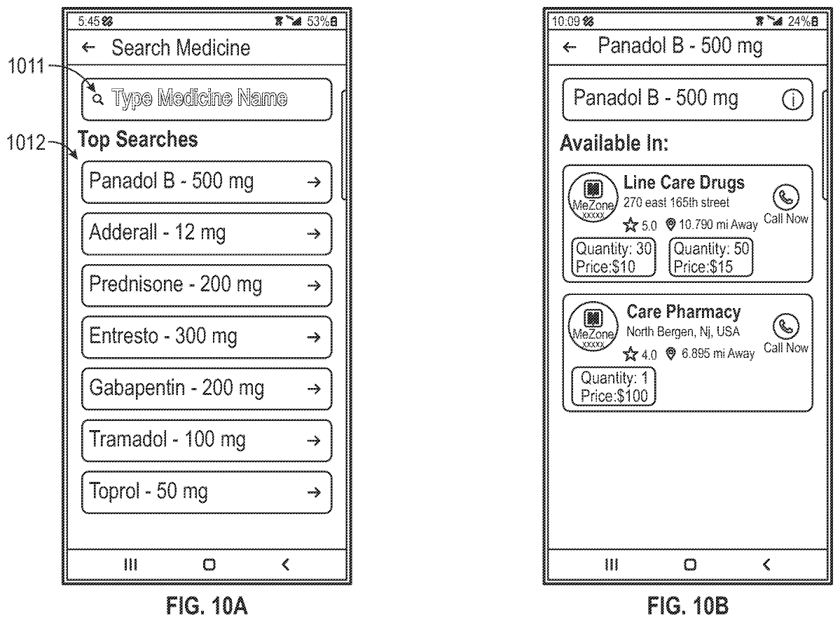

FIG. 10A and 10B illustrate an example user interface on the device 210 for searching medicine. FIG. 10A displays both search field 1011 and the recommended (most searched) medicine 1012. A user may either select one of the medicines in the list 1012 or enter a desired medicine in the text field 1011. On either of the action, The device 210 displays The list of pharmacy that has the stock of the selected medicine. FIG. 10B illustrates the respective list of pharmacy displayed in response to selection of medicine.

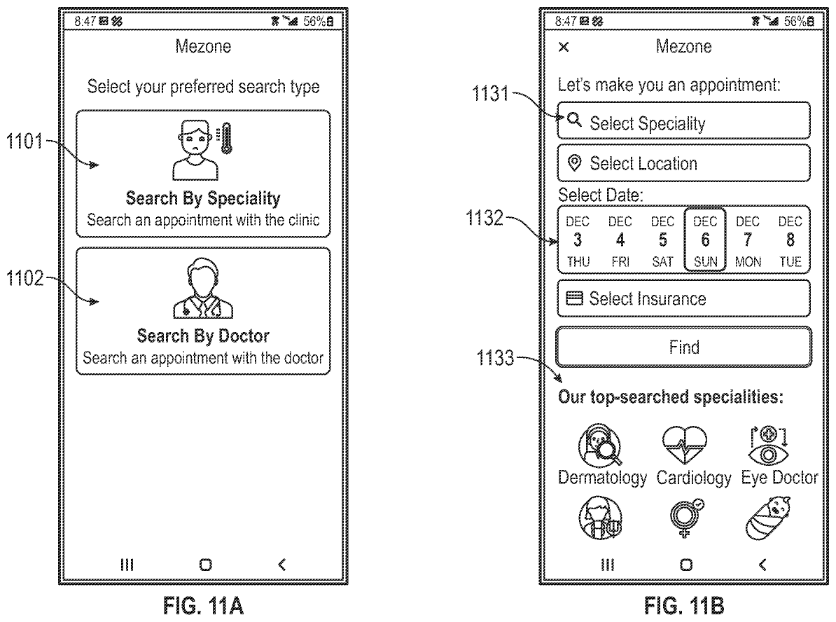

Similarly, FIG. 11A-11D illustrate the user interface on the device 210 enabling the search of doctors. As shown, in the FIG. 11A, the device 210 may present search user selection by specialty 1101 and search by doctor 1102. The selection of search by specialty may result with the user interface as shown in FIG. 11B and selection of and search by doctor 1102 may result The user interface as in FIG. 11C.

The user interface of FIG. 11B allows user to input the specialty in the field 1131, select date 1132, and also may list the proposed specialty 1133. User may provide the selection either typing the text in the field 1131 or selecting one from the recommendation 1133. User may additionally provide a location if intended location reference is not the current location.

Similarly, the user interface of FIG. 11C allows user to input the doctor name/partial name in the field 1141. The result of the search is presented as list 1142. User may select one of the doctors in the list 1141 for appointment. FIG. 11D represents the user interface on the device 210 when user selects one of the doctors in the list 1142. As shown, the user interface of FIG. 11D provides the details/profile 1151 of the doctor, personal information navigator 1152, review navigator 1153 and appointment selection button 1154.

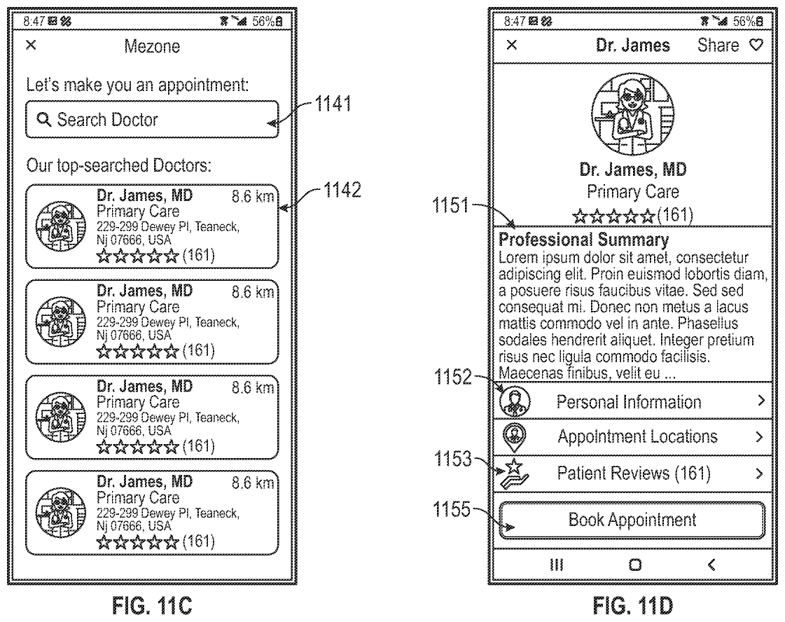

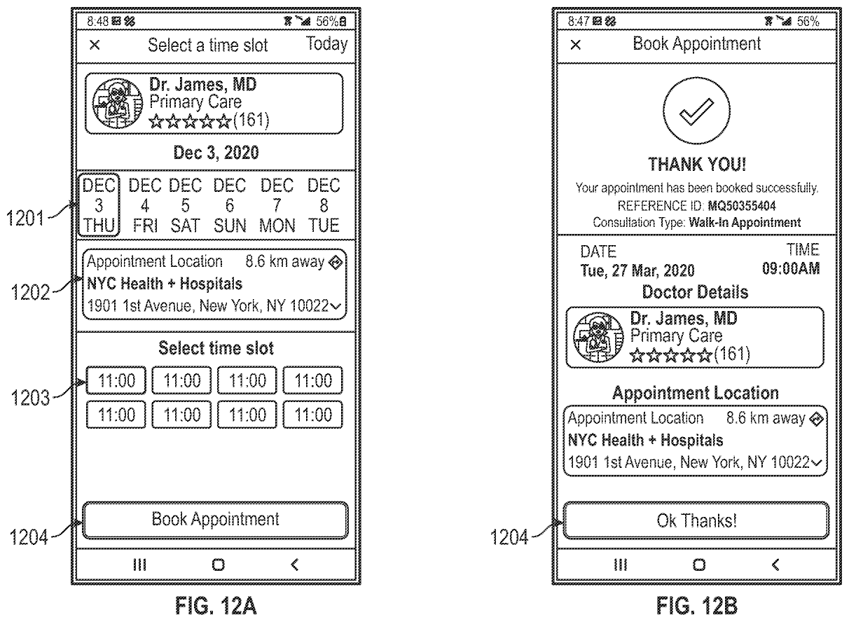

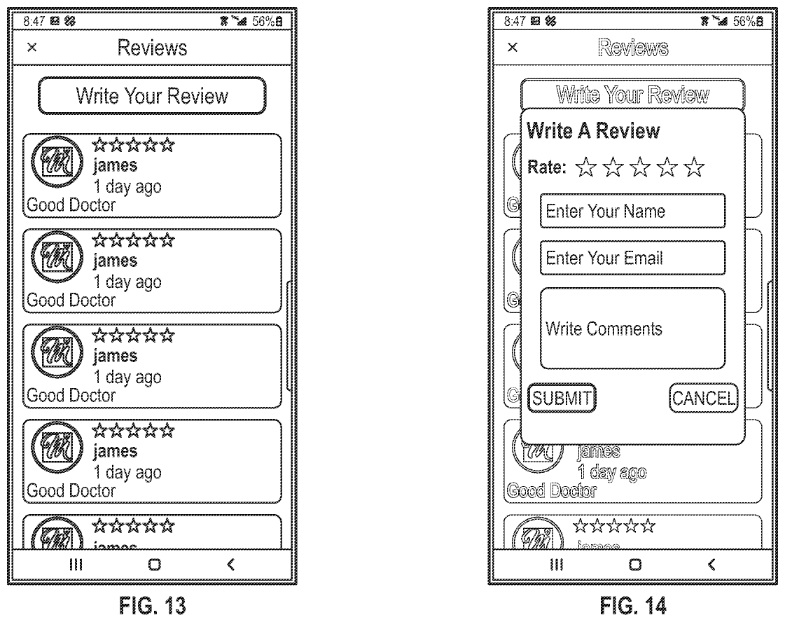

FIG. 12A and 12B illustrate the user interface for booking appointment and the confirmation respectively. The appointment with selected doctor is facilitated through date selection field 1201, location selection field 1202, time selection field 1203 and confirm appointment button 1204. FIGS. 13 and 14 illustrate the review and review input user interface respectively.

In operation, the device 210, 190 and 220-290 may operate to provide a workflow. For example, various embodiments provide a software application namely, The Medzone which allows a patient who is looking for a heath care provider such as a pharmacy, urgent care, hospital ER or nursing home near their home to locate same. Having located the health care provider and requested the service for example a prescription to be filled, in one embodiment The Medzone will require the pharmacy to deliver the medication the same day unless a cut off time is established, in that case the medication will be delivered the next day. If the primary Pharmacy cannot obtain the medication then a secondary pharmacy will be contacted to provide the service. The website and App will have a section under the category inventory, this section will be in real time. The pharmacy will be asked to give a real time inventory of the top 200 to 300 drugs, so when the patient goes to the inventory section and type the name of the drugs the pharmacy will show available in stock. The patient's doctor will send the electronic prescription to the pharmacy. The pharmacy will then deliver the medication to the patient the same day. In one embodiment, using The Medzone the doctor sends the electronic prescription via an encrypted transmission such as Blockchain. The pharmacy acknowledges receipt of the prescription. The receipt is time stamped and the consumer is notified. The consumer provides feedback about the day and time of delivery of the medication. The pharmacy also provides information about The Medzone shipment of the medication. Such information includes carrier, tracking number, day and time of shipment. In one embodiment, real time tracking of the shipment is made available to the consumer. Urgent Care and Hospital ER will also be listed on The Medzone's database. Again patient types in their zip code of their area of resident or another area they are currently, they need to see a doctor right away and need help locating a clinic which can see them without appointment. The list comes with Urgent Care facilities and Hospital ER in that area. The list will also include the specialty of the provider and their hours of operation, also will have a real time of waiting period to see a doctor at their facility. In one embodiment, the consumer/patient is provided with an appointment specifying the time slot allocated to the consumer/patient. Information such as insurance policy, driver's license number can be transmitted securely to the facility. Nursing Homes also fall under the category of health care providers. Medication is dispensed in nursing homes. As such, a prescription for a specific patient can be transmitted to a pharmacy following the same protocol as described above. There will be other categories for patient seeking help in the area of HIV services, needle exchange programs, opioids and harm reduction programs available at or near their location. Other tools will be available on the app and website to provide fast service to these customers. Machine Learning (ML) is used to implement these features and tools. The interface features several options arranged as pull-down menus in several pages. Some of these options include: Horse Screen, Registering for App, Search Bar, Search Features, Profile Features, Profile, and User Name log-in, Search Page, Search Page Features, Messaging, Transaction Features, Payment Features and Ask the Pharmacist section. The layout of the interface provides for ad space.

In addition to the user interface described in the above sections, the device 210 may be also configured with: (i) Home screen: Home Screen featuring search bar, messages, calendar, profile/business settings. Home screen will feature post of profiles that an individual chooses to “get connected” to. The calendar will allow for those who provide services to book appointments. A user can click a book on profile page and pick date for service. A payment link will be presented for payment. (ii) registration: registering for App. requires email address. Will give choice of creating business or just browsing/shopping. Then create your own Username/password. Profiles are available business/healthcare providers and every age group from pediatrics to geriatrics who are patients looking for alternative provider for their health care needs. Will give option to follow pages “get connected.” Coupons will offered to those who are registered. (iii) Search Bar: Search bar will allow search by country, county, state, city, and type of business/service provider. (App will be available to international market place). Business/service provider description details will allow a user to search by hash tags for greater detail. When creating business profile, the user will select a category but will also be able to have hash tags for greater details. (iv) Search Features: Type in category and or hash tags for greater details. (v) Profile features: Profiles will allow for customization. Will allow profile picture to be uploaded and used. Will allow people to post pictures and videos of services or goods to profile. Pictures and videos will allow for pay pal link to be present. If picture or video is lust ad for page no link for pay pal is required. Option to user. (vi) Profile: Profiles of business' will be able to be rated by the consumers. This will allow for business with low ratings to be avoided by users. Profiles main purpose will be shopping pages and will provided with contact information and booking information. (vii) User Name log-in. Consumers will have a Username and a password. Consumers will receive notification for purchases and transactions. Consumer will be responsible for making sure all payments were sent or received. (viii) Search Page. Search page will show listing of business/service providers and there rating. Description will be next profile icon. (ix) Search Page features. Will have the search engine features with home screen icon available at the bottom of screen. (x) Ask the Pharmacist. A person can ask a question on line and get a response from a pharmacist. But The Medzone is not a provider of healthcare. The Medzone will connect the patient to the provider of their request. What transpires between patient and provider is between both parties and The Medzone. (xi) Ad. revenue and ad. space.

While various embodiments of the present disclosure have been described above, it should be understood that they have been presented by, way of example only, and not limitation. Thus, the breadth and scope of the present disclosure should not be limited by any of the above-discussed embodiments but should be defined only in accordance with the following claims and their equivalents.

What claimed is,

1. A method performed on a computer system comprising:
receiving a Geographical location;
determining a geographical boundary in relation to the Geographical location;
listing a set of medical service providers within the boundary from a plurality of medical service providers;
displaying the set of medical service providers for selection; and
selecting a first medical service provider in the set of medical service provider;
connecting to the first medical service provider, wherein said connecting comprising one of a establishing communication, placing order, making an appointment, booking a facility.

2. The method of claim 1, wherein the computer system comprising a mobile device, a computer, a database, first server, a GIS server and an AI server
wherein the database comprising a plurality of data sets corresponding to the plurality of medical service providers, in that, each data set comprising at least one of a location information, services offered, stock of medicine, facilities, inventory, product information, and specialization.

3. The method of claim 2, wherein the plurality of medical service providers are grouped under plurality of categories comprising at least one of Pharmacy, doctors, hospital, laboratory, urgent care, harm reduction.

4. The method of claim 3, further comprising:
providing a user interface for searching a medicine;
presenting a list of preferred medicine and a text entry field to enter the medicine name;
selecting a first medicine from at least one of the list of preferred medicine and result of a search; and
listing a set of pharmacies having the first medicine in stock and located within the boundary;
selecting a first pharmacy from the set of pharmacies;
placing an order for the first medicine with the first pharmacy;
managing the order, said managing comprising at least one of a sending a order details to the first pharmacy, facilitating delivery of medicine.

5. The method of claim 4, wherein the set of preferred medicine is a frequently searched medicine.

6. The method of claim 3, further comprising:
providing a user interface for searching a first medical service provider in one of said category;
providing a list of preferred service providers;
presenting a list of category of medical service providers indicating a number of medical service providers within the geographical boundary;
selecting a first medical service provider category from the list of category;
presenting the first list of medical service provider of first category that are within the geographical boundary;
selecting a first medical service provider at least from the first list or from the list of preferred medical service provider;
placing a service request with first medical service provider, wherein said placing includes the selection of date, service type and location of service; and
booking the service request with the first medical service provider.

7. The method of claim 4, wherein the preferred medicine is determined by applying artificial intelligence on at least a history data, a user history, state of general medical situation and time.

8. The method of claim 6, wherein the list of preferred medical service provider is determined by applying artificial intelligence on at least a history data, a user history, state of general medical situation and time.

9. The method of claim 2, wherein the database comprising a plurality of doctors, each doctor comprising a data of at least one of name, address, location, specialization, consultancy locations, available time slots.

10. The method of claim 9, further comprising:
providing a user interface for searching a doctor;
providing a list of preferred doctor;
presenting a list of doctors within the geographical boundary;
presenting the list of doctors on a map;
selecting a first doctor in the list of doctors;
presenting the details of the first doctor;
placing an appointment request with the first doctor; and
recording a successful completion of the appointment.

11. The method of claim 2, further comprising receiving a review and displaying a ratings of first medical service provider, wherein the rating is determined based on the review.

12. A networked computer systems operative to provide medical services to a user comprising:
at least one mobile device configured to operate as user device;
a plurality of computers configured to operate as corresponding plurality of service providers;
a medical service facilitation server to facilitate medical services between the plurality of computers and the mobile device;
an Artificial intelligence server configured to perform tasks by applying logical reasoning that is similar to human logical reasoning;
a Machine learning engine configured to modify rules and conditions applied by the medical service facilitation server and Artificial intelligence server;
a GIS server operative to determine the location of the mobile device; and
a database configured to store datasets comprising at list one of products, services, facilities, availability, price, inventory, address, location, access information and any other information relevant for the services offered by the respective service providers;
a map server providing a first map in relation to the location of the mobile device
wherein, the medical service facilitation server presents a list of service providers on the mobile device based on the location of the mobile device.

13. The networked computer systems of claim 12, wherein the medical service facilitation server further comprising:
a location based health care/service provider module 410 operative to provide location based service provider access;
a medicine search module 420 operative to search medicine and its availability with the service providers;
a rating module 430 operative to receive reviews and provide ratings of the service provider;
a search doctor module 440 operative to search doctors by specialization and location;
a suggestion module 450 operative to generate suggestion and present the suggestion along with other services; and
an appointment booking module 460 operative to book appointments and facilities and monitor the same.

14. The networked computer systems of claim 13, wherein the mobile device further comprising:
a first user interface for receiving user location;
a second user interface for displaying a plurality of categories of the service providers;
a third user interface for displaying a first list of service providers on a map;
a fourth user interface for displaying the details of selected service providers;
a fifth user interface for listing a plurality of preferred medicine along with a search field for receiving a specific medicine;
a sixth user interface for listing a set of pharmacies offering the specific medicine
a seventh user interface for searching a specialty clinic and a doctor;
an eighth user interface for displaying the details of the doctor
a ninth user interface for booking an appointment with a doctor; and
a tenth user interface for confirming said appointment.

15. The networked computer systems of claim 14, wherein the medical service facilitation server comprising a web application interface (AAI) layers, plurality of services and a synchronizer, in that the web application interface (API) layers interact with the database through stored procedures.

16. The networked computer systems of claim 15, wherein the mobile device comprising a presentation layer having user interface, a business layer having application facade, workflow, components and model/entities and a data layer 740 having access to data, utility data and service tools.

17. The networked computer systems of claim 16, wherein the artificial intelligence engine is configured to recommend a list of top searched medicines based on the time, location, user history, current medical scenario in the locality/country.
